(19) United States
(12) Patent Application Publication
(10) Pub. No.: US 2009/0275802 A1
(43) Pub. Date: Nov. 5, 2009

(54) Distally Expandable Dilation Tube

(76) Inventors: David T. Hawkes, Pleasant Grove, UT (US); Thomas Sweeney, Sarasota, FL (US); Michael D. Ensign, Salt Lake City, UT (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 12/434,471

(22) Filed: May 1, 2009

Related U.S. Application Data
(60) Provisional application No. 61/049,628, filed on May 1, 2008.

(51) Int. Cl. A61B 1/32

(52) U.S. Cl. 600/219

(57) ABSTRACT

A transitional dilator including: a dilator body having a proximal end and a distal end, wherein the dilator body defines a sleeve orifice configured to receive an implant placement tube; and a round tip formed on the distal end of the dilator body, wherein the round tip defines a converging orifice terminating at an exit orifice configured to closely mate with a serial dilator; in which the round tip including a converging orifice further includes a wall split configured to receive the implant placement tube and expand to accommodate passage of the implant placement tube through the exit orifice.

### RELATED APPLICATIONS

The present application claims priority under 35 U.S.C. § 119(e) from the following previously-filed Provisional Patent Application, U.S. Application No. 61/049,628, filed May 1, 2008, entitled “Distally Expandable Dilation Tube,” and which is incorporated herein by reference in its entirety.

### BACKGROUND

Surgical procedures where medical devices need to be inserted into an incision in order to perform an operation can potentially be very invasive and extensive. Operations such as spinal surgery or similarly dangerous operations require adequate visibility and access to the area being operated on in order for the surgery to be safe and effective. In order to reduce post-operative healing time, scarring, and potentially other complications which may arise from making large incisions, minimally invasive techniques for inserting the instruments are desirable.

Consequently, medical professionals try to minimize complications such as those listed by making smaller incisions. Smaller incisions, however, make it more difficult to insert medical instruments and limit access and visibility within the incision. Because of this, there is a need in the field for a minimally invasive system and method for inserting medical instruments into a small incision without having a detrimental effect on the ability of a medical professional to effectively operate. Minimally invasive techniques can reduce the intra-operative damage and reduce the post-operative recovery time.

### SUMMARY

According to one embodiment of the present specification, a transitional dilator includes a dilator body having a proximal end and a distal end, in which the dilator body defines a sleeve orifice configured to receive an implant placement tube. A round tip is formed on the distal end of the dilator body, wherein the round tip defines a converging orifice terminating at an exit orifice configured to closely mate with a serial dilator. The round tip including a converging orifice further includes a wall split configured to receive the implant placement tube and expand to accommodate passage of the implant placement tube through the exit orifice.

In another exemplary embodiment, a method of inserting an implant placement tube includes: inserting a serial dilator in a wound; placing a transitional dilator over the serial dilator, wherein a tip of the transitional dilator is closely mated to the diameter of the serial dilator and configured to expand when forced open; inserting an implant placement tube into the transitional dilator; and forcing the placement tube through the tip of the transitional dilator, expanding the tip to displace surrounding soft tissue. The method may further include removing the serial dilator from the transitional dilator to create a pathway for implant placement.

### BRIEF DESCRIPTION OF THE DRAWINGS

The accompanying drawings illustrate various embodiments of the principles described herein and are a part of the specification. The illustrated embodiments are merely examples and do not limit the scope of the claims.

FIG. 1 is a perspective view of a serial dilator, according to the principles described herein.

FIG. 2 is a perspective view of a transitional dilator, according to principles described herein.

FIG. 3 is a perspective view of an implant placement tube, according to principles described herein.

FIG. 4 is a side view of a serial dilator, according to principles described herein.

FIG. 5 is a side view of a serial dilator disposed within a transitional dilator, according to principles described herein.

FIG. 6 is a side view of an implant placement tube disposed within a transitional dilator, according to principles described herein.

FIG. 7 is a side view of a transitional dilator with an expanded exit orifice, according to principles described herein.

FIG. 8 is a flowchart of a method of inserting an implant placement tube, according to principles described herein.

Throughout the drawings, identical reference numbers designate similar, but not necessarily identical, elements.

### DETAILED DESCRIPTION

The present specification relates to dilators for use during surgical operations. Specifically, a transitional dilator used to transition between an initial serial dilator and an implant placement tube is described herein. According to one exemplary embodiment, the transitional dilator is configured to expand the wound into which the serial dilator was placed so that the implant placement tube may be easily placed without causing unnecessary damage to tissue. Once the transitional dilator is inserted, according to one exemplary embodiment, the serial dilator may be removed to create a pathway to allow for surgical instruments to be used in the area being operated on, and the placement tube may be used to retain the incision open while a medical implant is inserted.

As used in the present specification and in the appended claims, the term “dilator” will be broadly understood to mean an instrument or tool that is used to dilate or widen an opening such as a surgical incision or a wound. The dilator may accomplish this, according to one exemplary embodiment, by displacing the soft tissue surrounding the opening. Also as used in the present specification and in the appended claims, the terms “incision” and “wound” are to be broadly interpreted to include any incision, portal, access point, wound or other opening made in a body where the operation is to take place.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “an embodiment,” “an example” or similar language means that a particular feature, structure, or characteristic described in connection with the embodiment or example is included in at least that one embodiment, but not necessarily in other embodiments. The various instances of the phrase “in one embodiment” or similar phrases in various places in the specification are not necessarily all referring to the same embodiment.

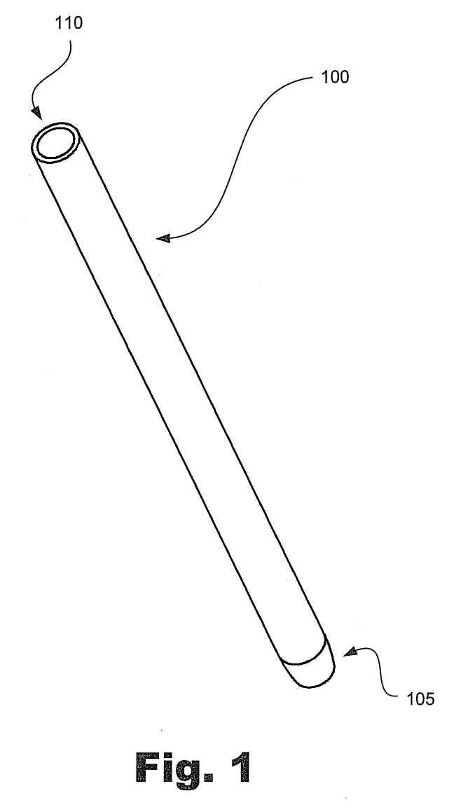

FIG. 1 is a perspective view of a typical serial dilator (100) which may be used to dilate a surgical opening or wound, according to one exemplary embodiment. As illustrated, the serial dilator (100) may include a tapered tip (105) having a smaller diameter at the very end of the serial dilator (100) and a larger diameter farther up the serial dilator (100) that allows the serial dilator (100) to be inserted into the wound and slowly displace the soft tissue around the wound as the serial dilator (100) is pushed farther into the wound, expanding the size of the wound while minimizing trauma to the tissues. Specifically, the gradual expansion caused by a serial dilator is configured to prevent ripping or tearing of skin and/or other tissue on a patient. Serial dilators (100) generally include a hollow barrel (110) that allows another tube or instrument to be inserted into the wound through the hollow barrel (110) of the serial dilator (100). However, according to the present exemplary system and method, solid serial dilators may also be incorporated by the present system. According to traditional use, the serial dilator (100) is held into place until the tube or other instrument is inserted into the wound. One example of an instrument that may be inserted into the wound through the serial dilator (100) is a suction tube for removing fluid from the wound.

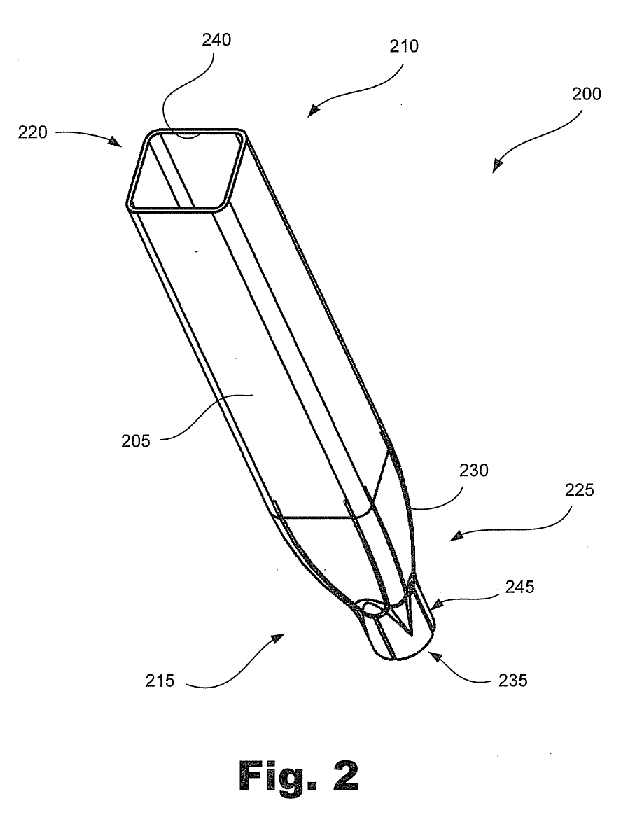

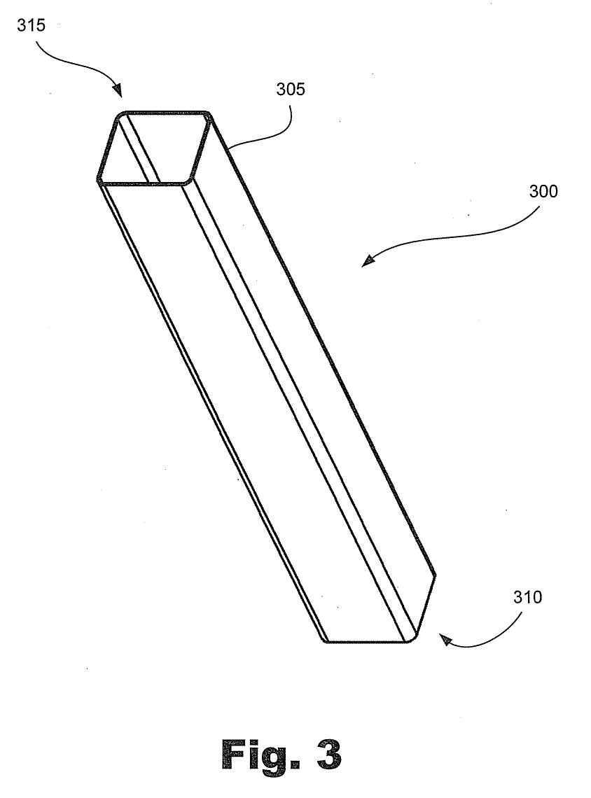

As used in the present specification, the serial dilator (100) may be used as an initial dilator for other procedures such as medical implants or the like, or alternatively may serve a plurality of purposes by providing a suction access in addition to the dilation functionality. The embodiment of FIG. 2 shows an exemplary transitional dilator (200) that may be inserted into a wound or surgical site after insertion of the serial dilator (100). The transitional dilator (200) is helpful for transitioning between the serial dilator (100) and a larger instrument to be inserted after the serial dilator (100), for example an implant placement tube (300) as shown in FIG. 3.

The exemplary transitional dilator (200) illustrated in FIG. 2 includes a main body (205) with a proximal end (210) and a distal end (215). According to one embodiment, the transitional dilator (200) has a sleeve orifice (220) defined by the main body (205) located at the proximal end (210) of the dilator (200). According to one exemplary embodiment, the sleeve orifice (220) defined by the main body (205) is configured to receive any number of implant placement tubes (300). According to one exemplary embodiment, the sleeve orifice (220) is sized to freely receive an implant placement tube while providing for easy translation of the implant placement tube with in the sleeve orifice to facilitate placement of the implant placement tube.

According to the exemplary embodiment illustrated in FIG. 2, a round tip (225) having a circular cross-section is also formed on the distal end (215) of the dilator body (205) that defines a converging orifice (230) and terminating at an exit orifice (235). The exit orifice (235) is configured to closely mate with the serial dilator (100) when the transitional dilator (200) is placed over the serial dilator (100). The exit orifice (235) has a circular shape and inner perimeter to allow the exit orifice to closely mate with and translate along a serial dilator (100), similar to that illustrated in FIG. 1. By closely mating with the serial dilator (100), the exit orifice (235) is able to be inserted into the wound via translation without causing unnecessary damage to the surrounding soft tissue.

The implant placement tube (300), according to one exemplary embodiment, is a hollow structure with an outer perimeter (305) measuring less than an inner perimeter (240) of the sleeve orifice (220) of the transitional dilator (200) so that the implant placement tube (300) may be inserted into the transitional dilator (200) via the sleeve orifice (220). While a square shaped implant placement tube (300) is illustrated in FIG. 3, it will be understood that an implant placement tube having any number of cross-sectional shapes may be incorporated by the present exemplary system. That is, a translating connection may be designed between the dilator body (205) having a corresponding cross-sectional shape and any number of placement tubes. As illustrated in FIG. 2, the dilator body (205) may have a constant inner perimeter (240) along a length of the dilator body (205) from the proximal end (210) until the round tip (225) at the distal end (215), at which point the inner perimeter (240) of the dilator body (205) converges to the exit orifice (235). The implant placement tube (300) may also have a constant outer perimeter (305) along a length of the tube (300) so that a distal orifice (310) of the tube (300) has the same outer perimeter as a proximal orifice (315) of the tube (300). In other embodiments, the transitional dilator (200) may have an inner perimeter (240) that tapers along the length of the dilator body (205). In such an embodiment, the implant placement tube (300) may also have an outer diameter (305) that tapers along the length of the tube (300) such that the tube (300) may be pushed into the sleeve orifice (220) of the transitional dilator (200) and through the exit orifice (235).

As the implant placement tube (300) is inserted into and pushed through the transitional dilator (200), the tube reaches the round tip (225) where the inner perimeter (240) of the dilator body (205) begins to converge to the smaller perimeter of the exit orifice (235). The round tip (225) of the dilator (200) has at least one wall split (245) that allows the inner diameter of the round tip (225) to be expanded, such that as the implant placement tube (300) is pushed through the transitional dilator (200), the act of pushing the tube (300) through the round tip (225) causes the exit orifice (235) to expand to allow further penetration of the tube (300). The exemplary transitional dilator (200) of FIG. 2 includes a plurality of wall splits (245) configured to allow the converging orifice (230) and exit orifice (235) to expand to receive the implant placement tube (300). When the placement tube (300) engages the converging walls of the round tip (225) and begins to impart an outward force on the walls, the wall splits (245) allow sections of the round tip (225) to separate from one another. The converging orifice (230) of the round tip in this embodiment includes a plurality of wall splits at the exit orifice (235). Consequently, the converging orifice (230) has more individual sections that are able to separate from one another in an expanded state, as explained below.

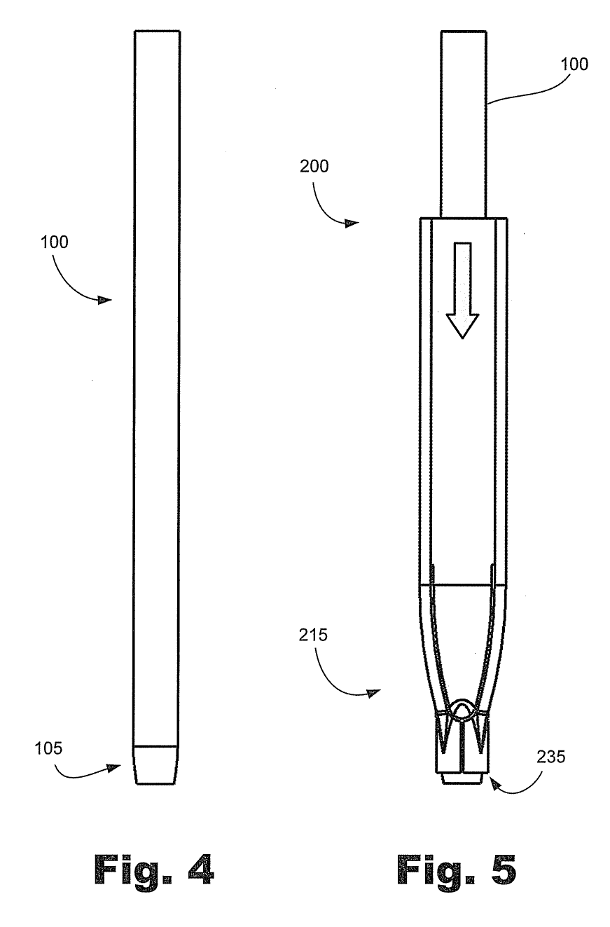

Use of the transitional dilator (200) in conjunction with a serial dilator (100) and an implant placement tube (300) is shown in greater detail in FIGS. 4-7. Specifically, FIG. 4 is a serial dilator (100) which may be inserted into a small incision (not shown) or portal to begin increasing the size of the incision opening. The serial dilator (100) has a tapered tip (105) to allow the serial dilator (100) to be inserted into the incision more easily. After the serial tip (105) is inserted into the incision, the transitional dilator (200) is placed over the serial dilator (100), as shown in FIG. 5. As mentioned, the distal end (215) of the transitional dilator (200) is placed over the serial dilator (100) first so that the exit orifice (235) of the transitional dilator (200) is proximate the incision. The circular shape of the exit orifice (235) and the close mating of the exit orifice (235) with the serial dilator (100) allow the exit orifice (235) to be translated along the serial dilator and inserted into the incision with the serial dilator (100), initially expanding the opening of the incision only slightly.

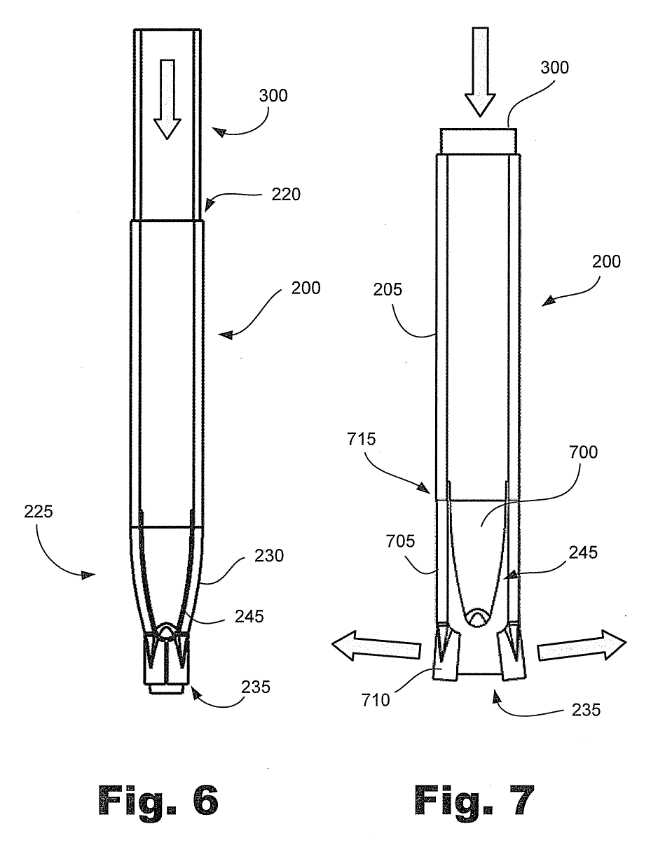

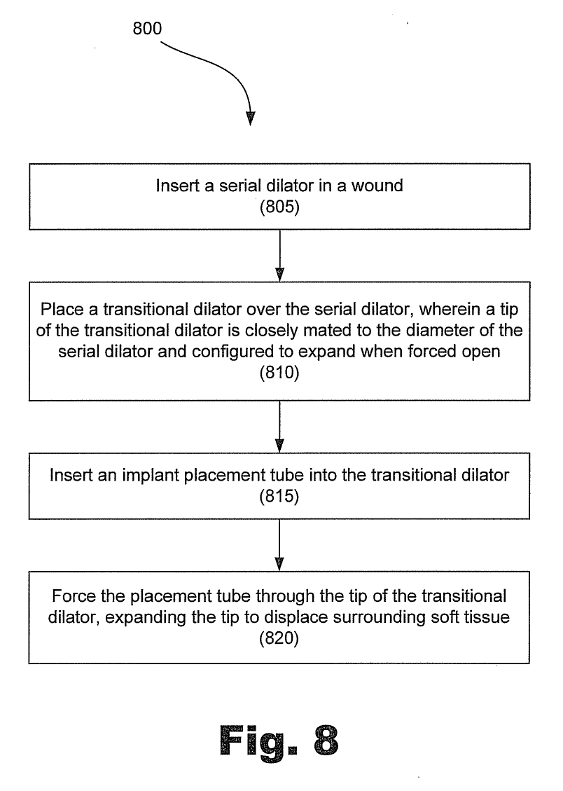

Once the exit orifice (235) of the transitional dilator (200) has been inserted into the incision, the implant placement tube (300) is inserted into the sleeve orifice (220) of the transitional dilator (200), as depicted in FIG. 6, and pushed through the transitional dilator (200). When the implant placement tube (300) reaches the round tip (225) and begins to move through the converging orifice (230) of the transitional dilator (200), the distal portion of the placement tube contacts the converging inner wall of the converging orifice (230). Consequently, the outer perimeter (305) of the implant placement tube (300) presses against the smaller inner perimeter of the converging orifice (230), causing an outward force to be applied to the round tip (225). As noted above, the round tip (225) has at least one wall split (245) through its perimeter to allow for the converging orifice (230) and exit orifice (235) to expand. Therefore, the implant placement tube (300) can be pushed completely through the transitional dilator (300), resulting in an expansion of the distal end (215) of the transitional dilator, as shown in the embodiment of FIG. 7.

According to one exemplary embodiment illustrated herein, the converging orifice (230) includes four elastic wall flaps (700) and four elastic corner flaps (705), each corner flap (705) being connected to a rounded section (710) of the exit orifice (235). The rounded sections (710) of the exit orifice (235) form a closed circular shape when in a non-expanded state. When the round tip (225) is in an expanded state, the rounded sections (710) are displaced away from each other due to the outward force from the implant placement tube (300). According to one exemplary embodiment, the round tip (225) is made from an elastically deformable material to allow the wall flaps (700) and corner flaps (705) to straighten and extend away from one another and radially outward from the center of the exit orifice. In the expanded state, the inner perimeter of the converging orifice (230) and the exit orifice (235) is increased to greater than the outer perimeter (305) of the implant placement tube (300) so that the implant placement tube (300) can be pushed all the way through the transitional dilator and into the surgical site of the wound. As the exit orifice (235) is moved into an expanded state, the rounded sections (710) push outward against the soft tissue surrounding the exit orifice (235) and expand the opening of the incision. Additionally, the transitional dilator (200) is shaped such that as the exit orifice (235) expands, the rounded sections (710) also push slightly upward, which may help hold the dilator (200) within the wound and prevent the dilator from slipping out during the operation.

When the incision has been expanded sufficiently and the exit orifice (235) is in its expanded state, the implant placement tube (300) may be placed in the incision. Expanding the incision will allow a surgeon to more easily see the operating area and use operating instruments in the area by providing light, irrigation, suction, and various medical instruments. Also, the implant placement tube (300) may have a sufficient inner perimeter to allow a surgeon to place larger items in the operating area without the need to make a longer incision. The serial dilator (100) may be removed from the incision and the transitional dilator (200) once the implant placement tube (300) is in place to clear the way for any instruments or for the implantation of other medical devices.

According to one exemplary embodiment, the round tip (225) may be integrally formed with the rest of the dilator body (205). The dilator body (205) may include a compliant hinged connection (715) with the round tip (225) at a transition area where the inner perimeter (240) of the transitional dilator (200) begins to converge. The compliant hinged connection (715) made be made by creating a wall thickness of the dilator body (205) that is thinner at the transition area than at the rest of the dilator body (205), thus allowing the wall and corner flaps (700, 705) to bend outward by flexing at the thinner wall thickness. An integrally formed dilator body (205), including the round tip (225), may allow for the transitional dilator (200) to be formed from a single mold or using a simple process. Alternatively, the round tip (225) may be coupled to the dilator body (205) by any number of hinge features including, but in no way limited to, a pined hinge, a flap hinge, and the like.

As illustrated, the wall splits (245) may extend past the hinged connection (715) or transition area and up into the main body portion. Extending the splits as such would allow portions of the round tip (225) to extend farther radially outward without putting too much strain on the hinged connection (715) or material from which the dilator (200) is made.

According to another embodiment, the round tip (225) may be attached to the rest of the dilator body by a separate hinged connection. The hinged connection may be such that the portion of the dilator body with a constant outer perimeter is formed separately from that of the round tip, and the round tip is connected after manufacture by a hinge piece that allows the wall flaps and the corner flaps to bend outward when a force is applied from within the dilator body. The hinge piece may be spring loaded such that in a resting state the exit orifice has a tight circular shape and closely mates the serial dilator when first installed.

The placement tube (300) may also be anchored to the transitional dilator (200) to prevent movement with respect to the transitional dilator once the tube has been inserted. The tube may be anchored using a spike or other protrusion at the distal end of the tube that hooks onto or is inserted into a portion of the dilator, preventing longitudinal movement of the tube. The tube may alternatively be configured to allow a modular spike or other attachment to be connected to both the placement tube and the dilator to anchor the tube and dilator to one another.

The transitional dilator (200) may be any shape suitable to receive a like-shaped implant replacement tube (300), or any such tube shaped so that the dilator holds the tube in place laterally and guides the tube into the incision. The dilator may be rectangular at the sleeve orifice (220) and the tube may be rectangular to fit within the dilator. The dilator may alternatively be square at the sleeve orifice, with the tube also being square so that the tube fits within the dilator. The exit orifice (235) is preferably the same shape as the serial dilator so that the exit orifice closely mates with the serial dilator (100) regardless of the orientation of either the transitional dilator or the serial dilator.

Other variations of the present specification may include a transitional dilator (200) that is made from multiple materials so that when the exit orifice (225) expands, the wall flaps (700) and corner flaps (705) are still connected by a second, stretchable material. The converging orifice of the round tip may have fewer or more flaps than shown in the figures, and the wall splits (245) may be located and designed in any fashion suitable for operating under the principles described herein.

When the operation or implantation is finished, the transitional dilator (200) and the implant placement tube (300) may be easily removed and disposed of. The dilators and tube are made from sterilized materials that prevent contamination in the incised area. The materials may also be made of disposable/recyclable materials for single-use to maintain a sterile operating environment. The transitional dilator, serial dilator, and placement tube may be made using inexpensive materials and mass-produced to further reduce costs.

In accordance with the transitional dilator described herein, a method (800) of inserting an implant placement tube includes first inserting (805) a serial dilator in a wound. After the serial dilator has been inserted in the wound to sufficiently expand the opening of the wound, a transitional dilator is placed (810) over the serial dilator, wherein a tip of the transitional dilator is closely mated to the diameter of the serial dilator and is configured to expand when forced open. An implant placement tube is then inserted (815) into the transitional dilator. The placement tube is forced (820) through the tip of the transitional dilator, expanding the tip to displace surrounding soft tissue. The method may also include removing the serial dilator to create a pathway for implant placement, as well as to provide visibility, light, and room for other medical instruments.

In another exemplary embodiment, the method further includes anchoring the implant placement tube to the transitional dilator after inserting the tube into the dilator and into the wound. The implant placement tube may be anchored to the dilator with a spike or protrusion formed on the distal end of the tube. Alternatively, a modular protrusion may be attached separately to both the dilator and the placement tube at any location in order to stabilize the tube and dilator and to prevent the tube from moving within the dilator.

The preceding description has been presented only to illustrate and describe embodiments of the invention. It is not intended to be exhaustive or to limit the invention to any precise form disclosed. Many modifications and variations are possible in light of the above teaching. Modification of the above disclosure may include only pieces of the system as exclusive members or combinations of pieces to form a useful application in which various described elements of the disclosure are not incorporated. As specific embodiments of the system and method have been described, it is important to recognize that each embodiment may be used alone or in conjunction pieces of the other embodiments.

What is claimed is:

1. A transitional dilator comprising:
a dilator body having a proximal end and a distal end, wherein said dilator body defines a sleeve orifice configured to receive an implant placement tube; and
a round tip formed on said distal end of said dilator body, wherein said round tip defines a converging orifice terminating at an exit orifice configured to closely mate with a serial dilator;
in which said round tip including a converging orifice further includes a wall split configured to receive said implant placement tube and expand to accommodate passage of said implant placement tube through said exit orifice.

2. The transitional dilator of claim 1, in which said sleeve orifice comprises a rectangular orifice and said exit orifice comprises a circular orifice.

3. The transitional dilator of claim 1, in which said sleeve orifice comprises a square orifice and said exit orifice comprises a circular orifice.

4. The transitional dilator of claim 1, in which said implant placement tube comprises a square perimeter shape.

5. The transitional dilator of claim 1, in which said implant placement tube comprises a rectangular perimeter shape.

6. The transitional dilator of claim 1, in which said implant placement tube comprises a perimeter shape configured to force said converging orifice open as said implant placement tube moves toward said distal end.

7. The transitional dilator of claim 1, in which said implant placement tube comprises a distal protrusion configured to anchor said implant placement tube to said transitional dilator after insertion.

8. The transitional dilator of claim 1, in which said implant placement tube is configured to provide for attachment of a modular spike at a distal end of said implant placement tube in order to anchor said implant placement tube to said transitional dilator after insertion.

9. The transitional dilator of claim 1, in which said round tip is made from an elastically deformable material.

10. The transitional dilator of claim 1, in which said round tip comprises a hinged connection to said dilator body.

11. The transitional dilator of claim 10, in which said hinged connection is a compliant hinge contiguous with said dilator body, wherein a thickness of said hinged connection is thinner than a thickness of said dilator body.

12. A method of inserting an implant placement tube, comprising:
inserting a serial dilator in a wound;
placing a transitional dilator over said serial dilator, wherein a tip of said transitional dilator is closely mated to the diameter of said serial dilator and configured to expand when forced open;
inserting an implant placement tube into said transitional dilator; and
forcing said placement tube through said tip of said transitional dilator, expanding said tip to displace surrounding soft tissue.

13. The method of claim 12, further comprising removing said serial dilator to create a pathway for implant placement.

14. The method of claim 12, further comprising anchoring said implant placement tube to said transitional dilator after insertion.

15. The method of claim 14, in which said implant placement tube comprises a distal protrusion configured to allow said anchoring.

16. A transitional dilator comprising:
a dilator body having a proximal end and a distal end, wherein said dilator body defines a sleeve orifice configured to receive an implant placement tube; and
a round tip formed on said distal end of said dilator body, wherein said round tip defines a converging orifice terminating at an exit orifice configured to closely mate with a serial dilator;
in which said round tip including a converging orifice further includes a wall split configured to receive said implant placement tube and expand to accommodate passage of said implant placement tube through said exit orifice;
wherein said sleeve orifice comprises a rectangular orifice and said exit orifice comprises a circular orifice; and
wherein said round tip is made from an elastically deformable material that is hingedly coupled to said dilator body.

17. The transitional dilator of claim 16, in which said implant placement tube comprises a perimeter shape configured to force said converging orifice open as said implant placement tube moves toward said distal end.

18. The transitional dilator of claim 16, in which said implant placement tube comprises a distal protrusion configured to anchor said implant placement tube to said transitional dilator after insertion.

19. The transitional dilator of claim 16, in which said implant placement tube is configured to provide for attachment of a modular spike at a distal end of said implant placement tube in order to anchor said implant placement tube to said transitional dilator after insertion.

20. The transitional dilator of claim 16, wherein said hinged connection comprises a compliant hinge contiguous with said dilator body, wherein a thickness of said hinged connection is thinner than a thickness of said dilator body.
